(19) United States
(12) Patent Application Publication
(10) Pub. No.: US 2026/0290571 A1
(43) Pub. Date: Sep. 24, 2026

(54) SYSTEM AND METHODS FOR MONITORING HEART FAILURE CONDITIONS USING A DYNAMIC PREDICTION INDEX

(71) Applicant: Impulse Dynamics NV, Willemstad (CW)

(72) Inventors: David PRUTCHI, Voorhees, NJ (US); Simeon Ioannis KEDIKOGLOU, Penn Valley, PA (US); Tamir BEN DAVID, Tel-Aviv (IL)

(73) Assignee: Impulse Dynamics NV, Willemstad (CW)

(21) Appl. No.: 19/474,287

(22) PCT Filed: Mar. 14, 2024
(86) PCT No.: PCT/IB2024/052483

§ 371 Date: Oct. 10, 2025

Related U.S. Application Data
(60) Provisional application No. 63/458,467, filed on Apr. 11, 2023.

(51) Int. Cl. G16H 40/63

(52) U.S. Cl. CPC G16H 40/63

(57) ABSTRACT

The present invention, in some embodiments thereof, relates to means and methods for monitoring heart failure (HF) conditions, more particularly, but not exclusively, to means and methods for monitoring HF by using a dynamic prediction index.

The present invention, in some embodiments thereof, relates to means and methods for monitoring heart failure (HF) conditions, more particularly, but not exclusively, to means and methods for monitoring HF by using a dynamic prediction index.

### RELATED APPLICATION/S

This application claims the benefit of priority of U.S. Provisional Patent Application No. 63/458,467 filed on 11 Apr. 2023, the contents of which are incorporated herein by reference in their entirety.

### FIELD AND BACKGROUND OF THE INVENTION

The present invention, in some embodiments thereof, relates to means and methods for monitoring heart failure (HF) conditions, more particularly, but not exclusively, to means and methods for monitoring HF by using a dynamic prediction index.

Implantable therapy devices for HF usually comprise several treatment alternatives, including for example Cardiac Resynchronization Therapy (CRT) and Cardiac Contractility Modulation (CCM). Such devices typically include sensing elements, means for collecting patient clinical data during the device operation and means for evaluating device operation parameters. Typical sensors may include Electrocardiogram (ECG) sensor, Bio-impedance sensor, activity sensor, position sensor, pressure sensor and temperature sensor. Additionally, medical data of the patient is collected, for example:

Patient medical history such as hospitalization and co-morbidities data, patient's physical parameters such as body weight, height, waist circumference and gait parameters.

Clinical parameters such as blood pressure, blood markers, blood oxidation levels, respiratory condition, action potential conduction velocity between two or more cardiac leads, lead's internal impedance, impedance between two or more cardiac leads, cardiac demand parameters_such as: average heart rate (HR) or Heart Rate Variability (HRV) during a set time period, cardiac abnormalities, including: atrial tachycardia rate, Premature Ventricular Contractions (PVC) rate, cardiac conditions including: historical thoracic impedance value and HF score.

Device operation parameters may include operation percentage, for example, number of heart beats having CRT or CCM stimulation during the active treatment periods, typically related as CRT percentage or CCM percentage.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. A system for monitoring a status of a user, comprising:
   a. an implantable device, configured to communicate with an external electronic device;
   b. an external electronic device, comprising:
      i. a generated reference database;
      ii. an assessment module comprising instructions to generate a personalized prediction index; and
      iii. a user database comprising user data.
Example 2. The system according to example 1, wherein said implantable device is an Implantable Cardioverter-Defibrillator (ICD).
Example 3. The system according to example 2, wherein said ICD is configured to perform one or more of monitoring the cardiac activity, providing Cardiac resynchronization therapy (CRT) and providing Cardiac contractility modulation (CCM).
Example 4. The system according to example 1, wherein said generated reference database is generated according to a method comprising:
   a. collecting historical data from a plurality of users; said historical data comprises a chronological timeline;
   b. indexing said collected data;
   c. analyzing said indexed data;
   d. grouping said analyzed indexed data according to similarities at a beginning of said chronological timeline;
   e. analyzing each of said analyzed groups to recover similarities and differences in the outcomes of said plurality of users;
   f. identifying diverging points in said chronological timeline;
   g. providing indexing values at said diverging points of said analyzed chronological timeline;
   h. storing said indexing values in a database for use as reference prediction index.
Example 5. The system according to example 1, wherein said personalized prediction index is generated according to a method comprising:
   a. collecting a plurality of data related to a plurality of different users;
   b. generating a plurality of reference prediction indexes based on said collected plurality of data;
   c. collecting data related to a determined user;
   d. comparing said collected data related to said determined user with said reference prediction indexes;
   e. generating a personalized prediction index according to said comparing.
Example 6. The system according to example 1, wherein said external electronic device is a server.
Example 7. The system according to example 1, wherein said external electronic device is an electronic device comprising instructions for delivering data to a server.
Example 8. The system according to example 1, wherein said external electronic device comprises a dedicated software comprising instructions to allow said user to insert user data.
Example 9. The system according to example 8, wherein said dedicated software comprises instructions for providing said user with a dedicated questioner.
Example 10. The system according to example 1, further comprising one or more external sources for providing data related to said user.
Example 11. The system according to example 1, wherein said personalized prediction index is used to predict an expected change in said status of said user.
Example 12. A method of generating a personalized prediction index, comprising:
   a. collecting a plurality of data related to a plurality of different users;
   b. generating a plurality of reference prediction indexes based on said collected plurality of data;
   c. collecting data related to a determined user;
   d. comparing said collected data related to said determined user with said reference prediction indexes;
   e. generating a personalized prediction index according to said comparing.
Example 13. The method according to example 12, wherein said data related to a determined user comprises one or more of user medical history, clinical parameters, data from an implantable device and data from external sensors.
Example 14. The method according to example 13, wherein said external sensors are one or more of ECG sensor, Blood pressure sensor, Bioimpedance sensor, Respiratory sensor, Movement sensor and Sleep sensor.
Example 15. The method according to example 12, wherein said collecting data related to a determined user comprises collecting data from one or more of user himself, one or more sensors connected to said user and one or more external sources.
Example 16. The method according to example 15, wherein said collecting data from the user himself comprises providing said user with a dedicated questioner.
Example 17. The method according to example 12, wherein said personalized prediction index is used to predict an expected change in a status of said user.
Example 18. A method of monitoring a status of a user, comprising:
   a. generating a personalized prediction index;
   b. collecting one or more status data of said user;
   c. updating said personalized prediction index according to said collected status data.
Example 19. The method according to example 18, wherein said generating a personalized prediction index comprises:
   a. collecting a plurality of data related to a plurality of different users;
   b. generating a plurality of reference prediction indexes based on said collected plurality of data;
   c. collecting data related to a determined user;
   d. comparing said collected data related to said determined user with said reference prediction indexes;
   e. generating a personalized prediction index according to said comparing.
Example 20. The method according to example 19, wherein said data related to a determined user comprises one or more of user medical history, clinical parameters, data from an implantable device and data from external sensors.
Example 21. The method according to example 20, wherein said external sensors are one or more of ECG sensor, Blood pressure sensor, Bioimpedance sensor, Respiratory sensor, Movement sensor and Sleep sensor.
Example 22. The method according to example 20, wherein said collecting data related to a determined user comprises collecting data from one or more of user himself, one or more sensors connected to said user and one or more external sources.
Example 23. The method according to example 22, wherein said collecting data from the user himself comprises providing said user with a dedicated questioner.
Example 24. The method according to example 18, wherein said personalized prediction index is used to predict an expected change in said status of said user.
Example 25. The method according to example 24, further comprising analyzing said collected data after said collecting and before said updating.
Example 26. The method according to example 25, wherein said analyzing comprises comparing said collected data with one or more reference data of one or more reference prediction indexes.
Example 27. The method according to example 18, further comprising updating at least one external entities with said updated personalized prediction index.
Example 28. The method according to example 18, further comprising sending an alert when a change in said personalized prediction index reaches a predetermined threshold.
Example 29. The method according to example 18, wherein said collecting one or more status data of said user comprises collecting data from one or more of user himself, one or more sensors connected to said user, one or more external sources.
Example 30. The method according to example 29, wherein said collecting data from the user himself comprises providing said user with a dedicated questioner.
Example 31. The method according to example 18, wherein said updating said personalized prediction index is used for predicting an expected change in said status of said user.
Example 32. A method of generating a reference prediction index database, comprising:
   a. collecting historical data from a plurality of users; said historical data comprises a chronological timeline;
   b. indexing said collected data;
   c. analyzing said indexed data;
   d. grouping said analyzed indexed data according to similarities at a beginning of said chronological timeline;
   e. analyzing each of said analyzed groups to recover similarities and differences in the outcomes of said plurality of users;
   f. identifying diverging points in said chronological timeline;
   g. providing indexing values at said diverging points of said analyzed chronological timeline;
   h. storing said indexing values in a database for use as reference prediction index.
Example 33. The method according to example 32, wherein said grouping at said beginning of said chronological timeline is done according to one or more of the following user related parameters: age, sex, medical condition, heart parameters, physiological parameters and medical history.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary system in relation to an exemplary implantable device, according to some embodiments of the invention;

FIG. 2 is a schematic representation of an exemplary principle of monitoring of the system, according to some embodiments of the invention;

FIG. 3 is a schematic representation of an exemplary system for monitoring the status of a patient, according to some embodiments of the invention;

FIG. 4 is an exemplary questioner shown in a personal device, according to some embodiments of the invention;

FIG. 5 is a flowchart of an exemplary method of monitoring a patient, according to some embodiments of the invention;

FIG. 6 is a flowchart of an exemplary method of generating a dedicated reference database, according to some embodiments of the invention; and

FIG. 7 is a schematic representation of an exemplary process of monitoring a patient, according to some embodiments of the invention.

### DESCRIPTION OF SPECIFIC EMBODIMENTS OF THE INVENTION

The present invention, in some embodiments thereof, relates to means and methods for monitoring heart failure (HF) conditions, more particularly, but not exclusively, to means and methods for monitoring HF by using a dynamic prediction index.

### Overview

An aspect of some embodiments of the invention relates to monitoring a status of a patient according to a predetermined set of parameters in order to allow estimation and/or possible prediction of a future (either probable or assured) change in the status of the patient. In some embodiments, the change in the status of the patient can be either an improvement in the status, a deterioration in the status. In some embodiments, the result of the monitoring might show a non-change in the status of the patient. In some embodiments, a personal prediction index is generated for each patient according to its own information. In some embodiments, each personal prediction index is compared to one or more similar prediction indexes stored in a dedicated database. In some embodiments, personal prediction indexes are updated according to new information relevant to that specific patient and then the new personal prediction index is newly compared to one or more similar prediction indexes stored in a dedicated database. In some embodiments, data generated by the comparison is used, in addition to provide an estimation and/or possible prediction of a change in the status of the patient, to assess possible treatments for the patient.

An aspect of some embodiments of the invention relates to a patient interactive software application (software app) configured to allow self-updating of a status of a patient used to complement data recovered from other sources to generate a personal prediction index. In some embodiments, the other sources are one or more of databases and data recovered form implanted devices.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

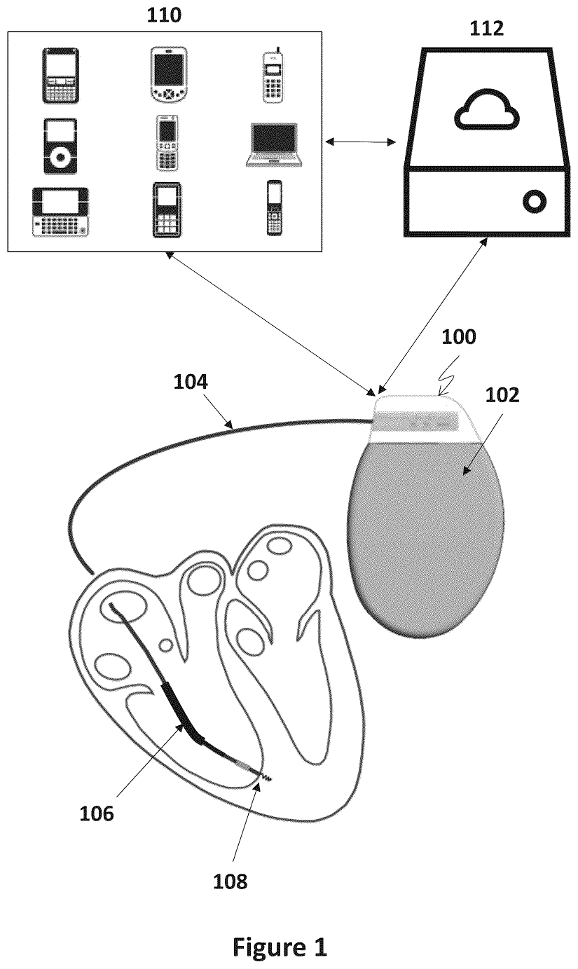

Referring now to FIG. 1, showing a schematic representation of an exemplary system in relation to an exemplary implantable device, according to some embodiments of the invention. In some embodiments, a system related to an exemplary implantable device comprises the implantable device 100 itself, one or more external devices 110 and at least one external server 112. In some embodiments, as will be further explained below, the implantable device 100 comprises dedicated hardware and software that allow two-way communication between the implantable device 100 and the one or more external devices 110 and/or the at least one external server 112. In some embodiments, the one or more external devices 110 and the at least one external server 112 communicate with each other.

In some embodiments, the implantable device 100 comprises a pulse generator comprising a housing 102 and one or more leads 104, in FIG. 1 only one lead 104 is shown, which are optionally couplable to the housing 102 of the device 100 with one or more connectors (not shown). In some embodiments, the housing 102 is implanted outside the heart, for example in the subclavian area. Optionally, implantation is via a minimally invasive procedure. In some embodiments, the housing 102 is implanted subcutaneously, in proximity of the left chest. In some embodiments, the housing 102 comprises within, all the necessary electronics and power source for the correct functioning of the implantable device 100, for example, a pulse generator is optionally used to generate the signal, for example, including a power circuitry, for example, including one or more storage capacitors. In some embodiments, the device 100 further comprises a ventricular detector used to detect atypical ventricular activation, which can be a contra-indication to signal application. In some embodiments, not shown in FIG. 1 since only one exemplary lead 104 is shown, the device 100 further comprises an atrial detector used to detect atypical atrial activation, which may be used as an input to decision making by device. In some embodiments, the device 100 further comprises one or more sensors configured to deliver one or more sensor input data, for example electrical sensors or other sensors, such as flow, pressure and/or acceleration sensors. In some embodiments, data from the sensors is optionally further processed (e.g., by a controller and/or detectors) and are optionally used as an input to decision making processes in device 100, which will be further explained below. In some embodiments, the device 100 further comprises a controller configured for executing one or more logics to decide, for example, a timing and/or other parameters of a signal and/or if a signal is to be applied, which will be further explained below. In some embodiments, the controller controls the applying of stimulation pulses according to the treatment plan. Optionally, the controller effects a change in the plan, for example so as to compensate for real time deviations from the treatment plan (e.g. a skipped stimulation). In some embodiments, the device 100 further comprises a memory configured for storing logic, past effects, therapeutic plans, adverse events and/or pulse parameters. In some embodiments, the controller and/or memory are programmed with one or more treatment plans (optionally set for the specific patient) and/or with one or more fallback treatment plans.

In some embodiments, the controller and/or memory are programmed with one or more instructions for monitoring a morphology of an ECG signal for managing the implantable device 100, for example an Implantable Cardioverter-Defibrillator (ICD).

In some embodiments, the device 100 further comprises a logger configured for storing activities of device 100 and/or of the patient. In some embodiments, such a log and/or programming may use a communication module to send data from device 100, for example, to a programmer (not shown) and/or a dedicated external device 110 like a mobile phone, tablet, etc., and/or an external server 112; and/or to receive data, for example, programming, for example, pulse parameters. In some embodiments, data is shared between all three: an external device, a server and an implanted device.

Following the exemplary option that the implantable device 100 is, for example, an Implantable Cardioverter-Defibrillator (ICD), a same lead 104 is used for all the necessary activities, for example, monitoring the cardiac activity, providing Cardiac resynchronization therapy (CRT), providing Cardiac contractility modulation (CCM), and any combination thereof. In some embodiments, regarding the activation and functioning of the ICD, activation of the ICD is by an ICD module which includes or is connected to: ICD control, a defibrillation pulse generator (via one or more capacitors), a power source (e.g. a battery) and power source management circuitry, and ICD sensor which senses an applied pulse to verify the pulse is within a selected (e.g. programmed) amplitude and/or duration. In some embodiments, activation of cardiac contractility modulation is by a cardiac contractility modulation module which includes or is connected to: cardiac contractility modulation control, a cardiac contractility modulation generator. As mentioned before, in some embodiments, the ICD coil and one more electrodes for pacing and/or cardiac contractility modulation are configured on the same lead. In some embodiments, separate leads are used for the ICD coil and one more electrodes for pacing and/or cardiac contractility modulation.

In some embodiments, the lead 104 extends from the housing 102, and at least a distal segment of the lead is implanted within the heart, as schematically shown for example in FIG. 1. In some embodiments, the lead comprise an ICD defibrillation coil 106 located along the lead 104. In some embodiments, the lead 104 ends with a tip electrode 108. In some embodiments, the tip electrode may be configured as a contact electrode, a screw-in electrode, a sutured electrode, a free-floating electrode and/or other types. In some embodiments, a tip electrode is formed with a threading so as to be threaded into the tissue. Alternatively, a tip electrode is solely placed in contact with the tissue.

In some embodiments, the cardiac contractility modulation signal is applied to the heart during a relative and/or absolute refractory period of the heart. In some embodiments, the signal is selected to increase the contractility of a cardiac ventricle when the electric field of the signal stimulates such ventricular tissue, for example, the left ventricle, the right ventricle and/or a ventricular septum. In some embodiments of the invention, contractility modulation is provided by phosphorylation of phospholamban caused by the signal. In some embodiments of the invention, contractility modulation is caused by a change in protein transcription and/or mRNA creation caused by the signal, optionally in the form of reversal of a fetal gene program. It is noted that in some embodiments the cardiac contractility modulation signal may be excitatory to tissue other than that to which it is applied.

While not being limited to a single pulse sequence, the term cardiac contractility modulation is used to describe any of a family of signals which includes a significant component applied during an absolute refractory period and which has a clinically significant effect on cardiac contractility in an acute and/or chronic fashion and/or which causes a reversal of fetal gene programs and/or which increases phosphorylation of phospholamban. In some embodiments, the signal is potentially excitatory to one part of the heart but non-excitatory to other parts. For example, a signal can be excitatory in atria, but applied at a timing (relative to ventricular activation) when it is not excitatory in the ventricle.

In some embodiments of the invention, the signal while potentially stimulatory during the receptive period of the cardiac cycle, is non-excitatory due to its timing. In particular, the signal is applied during the refractory period of the tissue which is affected by it and, optionally, within the absolute refractory period.

In some embodiment, a device electrode, for example the cardiac contractility modulation applying electrode, is used for measuring the R wave amplitude and/or RR interval of the cardiac cycle.

In some embodiments, the implantable device 100 comprises one or more sensors, for example, ECG sensor, Bio-impedance sensor, activity sensor, position sensor and pressure sensor.

### Exemplary Stimulations

In some embodiments, the exemplary device provides one or more of the following stimulations ICD, CCM and CRT.

### Exemplary Principle of the Monitoring System

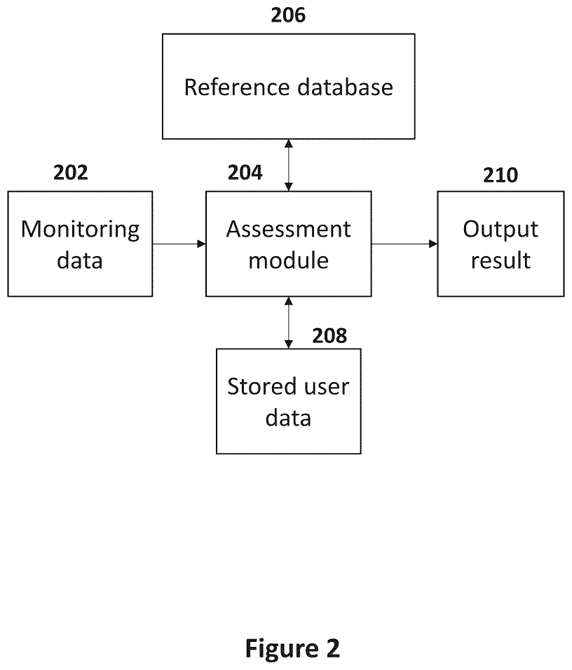

Referring now to FIG. 2, showing a schematic representation of an exemplary principle of monitoring of the system, according to some embodiments of the invention. In some embodiments, monitoring data 202 is provided into the system and/or is collected by one or more elements within the system (see FIG. 1), and more specifically, the data is provided to an assessment module 204 within the system. In some embodiments, the assessment module 204 utilizes data found in a reference database 206 and stored data related to the specific patient 208 to perform an assessment (which will be further explained below). In some embodiments, the results of the assessment are sent 210 to one or more entities outside the system (for example the patient, a physician, insurance company, etc.). In some embodiments, according to the results of the assessment, an alert is sent to one or more entities when the results of the assessments reach a predetermined threshold (see below for more explanations).

In the following paragraphs, a more detailed description of the monitoring system will be explained.

### Exemplary Monitoring of a Status of a Patient

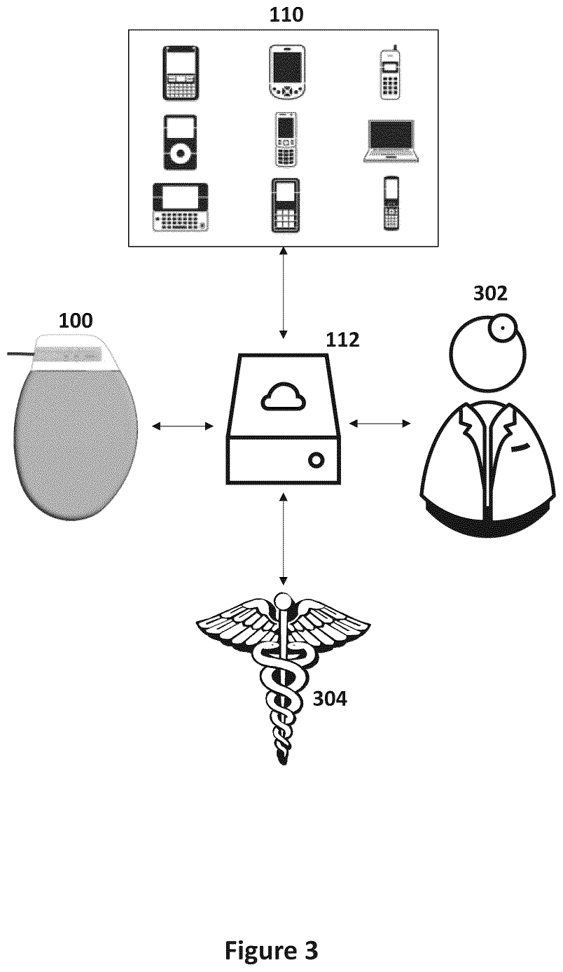

Referring now to FIG. 3, showing a schematic representation of an exemplary system for monitoring the status of a patient, according to some embodiments of the invention. Same reference numbers are kept for the same elements. In some embodiments, a personal status of a specific patient is monitored by the system utilizing one or more sources of information. In some embodiments, as mentioned above, information (data) is collected by the implantable device 100 and sent, for example, to a server 112 and/or an external device 110 (or to an external device 110 and then to a server 112 from that external device 110). In some embodiments, another source of information is data received from a physician and/or nurse 302. In some embodiments, another source of information is data received from a pharmacy 304. In some embodiments, the server comprises the assessment module 204 shown in FIG. 2.

In some embodiments, another source of information is the patient itself (not shown), which, in some embodiments, is provided with a dedicated external patient interactive software application (referred hereinafter as software app), configured to allow self-insertion of information that will be used for the monitoring of the status of the patient.

In some embodiments, exemplary data collected by the system can be one or more of:
1. Patient medical history: for example hospitalization and co-morbidities data, patient's physical parameters such as body weight, height, Waist circumference and gait parameters, which are stored as “stored user data 208” as shown in FIG. 2.
2. Clinical parameters: for example blood pressure, blood markers, blood oxidation levels, respiratory condition, action potential conduction velocity between two or more cardiac leads, leads internal impedance, impedance between two or more cardiac leads, cardiac demand parameters such as: average HR or HRV during a set time period, cardiac abnormalities, including: atrial tachycardia rate, PVC rate, cardiac condition including: historical thoracic impedance value and HF score (monitoring data 202 and/or stored user data 208 shown in FIG. 2).
3. Device operation parameters: for example operation percentages (for example the number of heart beats having CRT or CCM stimulation during the active treatment periods). In some embodiments, operation percentages are typically related as CRT percentage or CCM percentage (monitoring data 202 and/or stored user data 208 shown in FIG. 2).
4. External monitoring devices: for example, the user wears one or more sensors, optionally internal sensors (included in the implant), optionally external sensors, located in dedicated wearable devices. In some embodiments, the parameters monitored are provided to the system for continuous evaluation and generation of the personalized prediction index. In some embodiments, exemplary sensors are one or more of:
ECG sensor for detection of Heart rate/heart rate variability/arrhythmia events;
Blood pressure sensor for detection of systolic/diastolic BP in the RA/RV/LA/LV or arterial pressure;
Bioimpedance sensor for detection of lungs congestion;
Respiratory sensor for detection of respiratory rate and respiratory volume, breathing stop;
Movement sensor for detection of patient activity;
Sleep sensor for detection of sleep duration, sleep stages duration;
Temperature sensor for measuring body temperature.

### Exemplary External Patient Interactive Software App

In some embodiments, the system comprises a dedicated software configured to allow a user (patient) to insert personal information, for example health related information (but not only), in order to assist the system in monitoring the status of the user. In some embodiments, the personal information is automatically collected and stored by one or more external sources, for example, by implantable devices (where dedicated storage memory is provided for this role), physicians, pharmacies, mobile devices, smart watches, portable medical devices, etc. In some embodiments, exemplary data collected, manually and/or automatically, comprises one or more of: physiological data, including daily average activity level (for example, accelerometer-based), daily average posture (for example, accelerometer-based), daily average heart rate, daily average heart rate variability, VT/VF episodes (in implantable devices that incorporate ICD therapies), patient's demographics, quality-of-life data collected a Patient Health Survey administered through the software app (see below a table with an exemplary questioner for a patient).

### Exemplary Questioner for a Patient

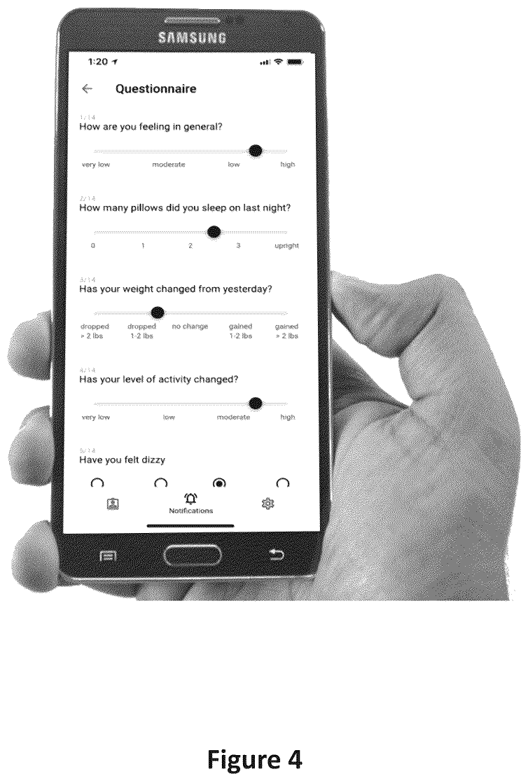

In some embodiments, the patient is provided with a dedicated questioner specifically designed to collect the relevant data that is used to assess the current status of the patient, which is then used to evaluate possible changes in the status of the patient. FIG. 4 shows an exemplary questioner shown in a personal device (mobile device).

In some embodiments, an exemplary questioner is as shown in the following table:

| Exemplary Questions | Exemplary Answer Choices |
|---|---|
| How are you feeling in general? | Visual scale 1 to 10 |
| How many pillows did you need to | 0, 1, 2, 3, sleep upright |
| sleep on last night? | |
| Has your weight changed from | (using scale) dropped >2 lbs., dropped 1-2 |
| yesterday? | lbs., no change, gained 1-2 lbs., gained >2 |
| | lbs. |
| Has your level of activity ability | (using scale) decreased significantly, |
| changed? | decreased mildly, no, increased mildly, |
| | increased significantly |
| How much activity have you | High (5 hours/day), moderate (3-5 |
| averaged in the past few days? | hours/day), low (1-2 hours/day), very low |
| | (<1 hour/day) |
| Have you felt dizzy? | No, yes but less than usual, yes same as usual, |
| | yes but more than usual |
| Are you feeling fatigued? | No, yes but less than usual, yes same as usual, |
| | yes but more than usual |
| Are you having difficulty climbing | No, yes but less than usual, yes same as usual, |
| the stairs? | yes but more than usual |
| Have you felt your heart racing? | No, yes but less than usual, yes same as usual, |
| | yes but more than usual |
| Have you felt your heart beating | No, yes but less than usual, yes same as usual, |
| irregularly? | yes but more than usual |
| Are you short of breath? | No, yes but less than usual, yes same as usual, |
| | yes but more than usual |
| Do you have swelling in your | No, yes but less than usual, yes same as usual, |
| ankles and legs? | yes but more than usual |
| Have you had any chest pain while | No, yes but less than usual, yes same as usual, |
| active? | yes but more than usual |
| Have you had any chest pain while | No, yes but less than usual, yes same as usual, |
| resting? | yes but more than usual |
| Have you missed any medications | Yes/No |
| in the last 24 hours? | |

It should be understood that a questioner may comprise more or less or different questions that those disclosed above, and that these were provided to allow a person having skills in the art to understand the invention.

### Detailed Explanations of Exemplary Monitoring of a Status of a Patient

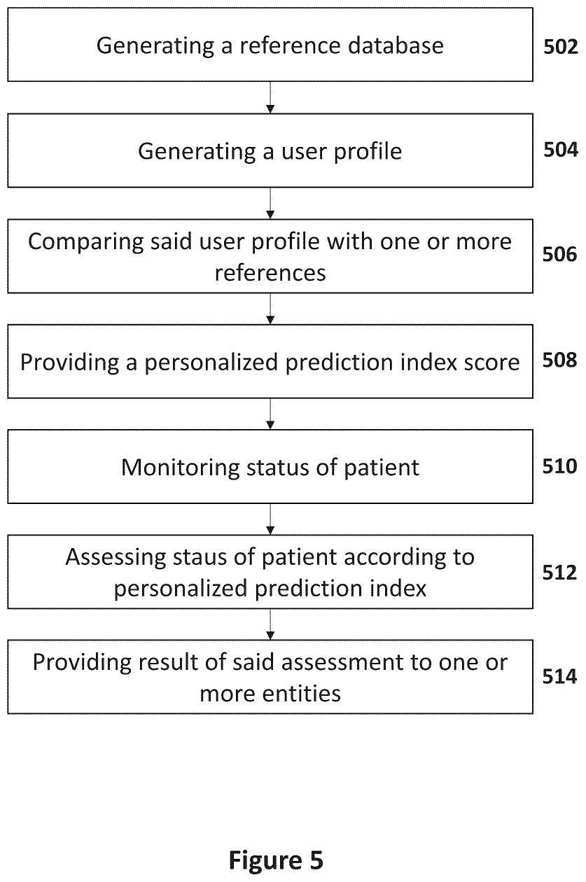

Referring now to FIG. 5, showing a flowchart of an exemplary method of monitoring a patient, according to some embodiments of the invention.

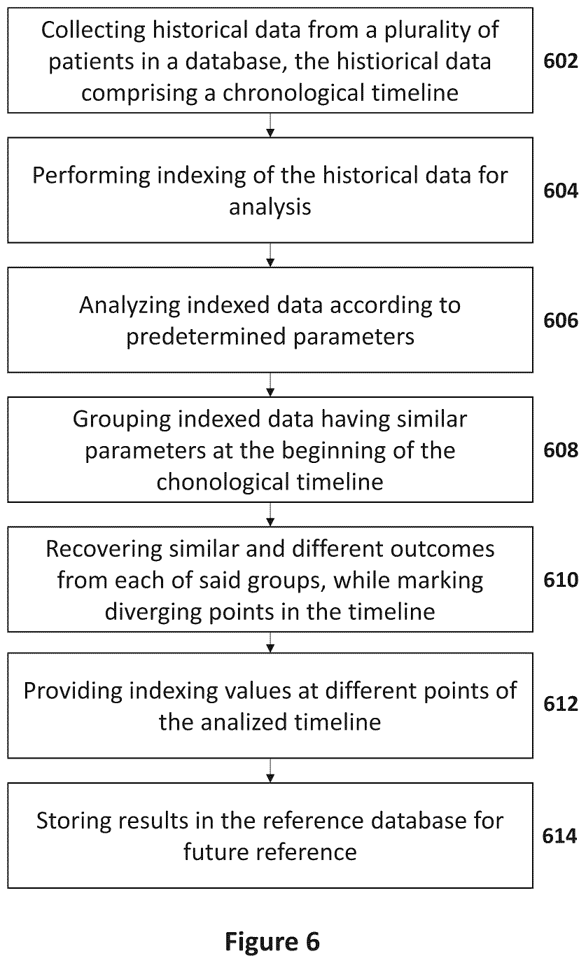

In some embodiments, a reference database is generated 502. In some embodiments, reference database is generated using for example historical data from a plurality of patients. See below regarding FIG. 6, showing a flowchart of an exemplary method of generating a reference database, according to some embodiments of the invention.

In some embodiments, a user profile is generated 504. In some embodiments, historical data related to the specific user/patient is generated. In some embodiments, the data is inserted by one or more of the patient, a physician, a pharmacist and any other dedicated authorized entity.

In some embodiments, the user profile is compared with one or more data in the reference database 506.

In some embodiments, based on the results of the comparison, a personalized prediction index is generated and provided 508 (see below).

In some embodiments, the system monitors the status of the patient 510, as explained elsewhere herein.

In some embodiments, the system is configured to assess the status of the patient using the data received from the monitoring in view of the personalized prediction index of the patient 512.

In some embodiments, the results of the assessments are then sent to one or more external entities, which use this information to make treatment decisions 514.

In some embodiments, when a change in the personalized prediction index reaches a predetermined threshold, the system is configured to send an alert to one or more entities.

Referring now to FIG. 6, showing a flowchart of an exemplary method of generating a dedicated reference database, according to some embodiments of the invention. In some embodiments, the system comprises a reference database that is used during the assessment of the status of the patients. In some embodiments, an exemplary method of generating a reference database is as follows:

In some embodiments, historical data is collected in a database 602. In some embodiments, the historical data comprises a chronological timeline of the data, which is used to analyze the data according to determined parameters. In some embodiments, historical data might be provided as “raw data”, and in some embodiments, the database is configured to organize the raw data and generate a chronological timeline of the historical data.

In some embodiments, indexing is performed on the historical data 604. In some embodiments, indexing is performed as known in the art, and it comprises, for example, providing internal tags to data in order to allow identification and organization of the data. In some embodiments, a potential advantage of indexing the data is that it facilitates the comparison calculations during the assessments.

In some embodiments, the indexed data is then analyzed 606 according to predetermined parameters.

In some embodiments, an exemplary predetermined parameters of analysis is data having similar data at a beginning of a timeline, for example, patients that presented for the first time heart failure. In some embodiments, further subgroups of predetermined parameters of analysis can be found, for example, patients that presented for the first time heart failure (HF) and have diabetes.

In some embodiments, indexed data having similar parameters at the beginning of the timeline are grouped together 608. In some embodiments, the grouping at the beginning of the chronological timeline is done according to one or more of the following user related parameters: age, sex, medical condition, heart parameters, physiological parameters and medical history.

In some embodiments, each group is then further analyzed to recover similarities and differences in the outcomes of the patients, while marking the diverging points in the timelines 610. For example, two different patients (patient A and patient B), both presented HF at the age of 45, no diabetes, at a relative time zero (TO) of the timeline. At relative time T5 (5 years after TO) patient A died and patient B did not. In some embodiments, the system is configured to analyze the data between T0 and T5 to find the diverging point(s).

In some embodiments, the system provides indexing values at diverging points of the analyzed times lines 612. In some embodiments, the indexing values are provided in order to provide a numeric value to a relative status of a patient at a specific time on the timeline, the time being relative to specific parameters (for example health parameters).

In some embodiments, the results are then stored in the reference database for future use and reference 614.

It should be understood that a reference database can be generated in other ways and using other steps. The abovementioned method has been provided to allow a person having skills in the art to understand the invention. The scope of the term “reference database” includes also reference databases generated by different methods but are used by the system in a similar manner.

### Exemplary Generation of User Profile

In some embodiments, the system is configured to collect all the relevant data related to a specific user and generate a user profile that will be used, at first, as the basis data profile for monitoring the status of the patient, and later (due to updates in the data of the user), as the up-to-date data profile of the patient used for assessments in the status of the patient. In some embodiments, exemplary data collected is as described herein elsewhere.

### Exemplary Generation of Personalized Prediction Index

In some embodiments, the system comprises instructions for generating a personalized prediction index for each patient/user.

In order to facilitate the explanation below, personal data will be referred just as “data”, but it should be understood that it refers to any kind of data as explained above. Additionally, a “prediction index” is calculated and/or re-calculated by comparing one or more changes in parameters recovered and/or derived from the “data”, as will be further explained below.

In some embodiments, once the user profile has been generated, the profile is compared with profiles stored in the reference database. In some embodiments, at the beginning of the monitoring, when the user profile has been just entered into the system, the system uses the newly user profile as starting point for the monitoring and the comparison is performed according to that starting point. For example, the starting point for a certain patient at TO is defined when the user profile is generated. The system compares that starting point with similar reference profiles within the reference database. In some embodiments, a starting point may match one or more reference profiles within the reference database. In some embodiments, the system uses the results of the comparison to provide a first personalized prediction index score to the user/patient. In some embodiments, at TO, since no new data has yet been entered into the system, the personalized prediction index score is more “flexible”, in the sense that many outcomes can happen according to the specific new data that will be entered. For example, at TO, the patient with no diabetes has just suffered a HF, and no new data has been entered yet, the first personalized prediction index score is for example “5”. This first personalized prediction index score matches many reference profiles having different outcomes (as explained above in relation with the generation of the reference database). As the time passes, for example at T2 (two years from TO), new data is inserted in the system that updates the user profile, for example, the patient developed diabetes. This new data “eliminates” non-relevant outcomes of reference profiles leaving much more relevant outcomes reference profiles, which are used for the calculation of the (now re-calculated) personalized prediction index score (now amended for example to “3”), and the assessment of the status of the user. In this example, developing diabetes is a factor that show a deterioration in the status of the patient, which, if not treated, might cause irreversible damage to the patient. This information is provided to external entities that will use this information to perform health-related decisions related to the specific patient.

In some embodiments, the system is configured to provide a “weight” to new entry data in order to facilitate the assessment of the state of the patient and the calculation of the new personalized index, as will be shown and explained for example below.

In some embodiments, for patients having the implantable device treatment such as CCM or CRT, which historical data has been collected, a dedicated set of parameters are set to generate a personalized prediction index. In some embodiments, a plurality of parameters are monitored, but only some parameters affect the personalized prediction index. In some embodiments, as mentioned above, exemplary condition can be: chances for hospitalization, level of HF decompensation, chances for cardiac pathology for example cardiac arrhythmia. In some embodiments, the personalized prediction index takes under consideration each specific condition. In some embodiments, alerts are provides whenever the personalized prediction index indicates higher chances for a condition to worsen.

### Exemplary Scale of the Personalized Prediction Index

In some embodiments, any type of scale of scale can be used to reflect a prediction index. For example, a numerical scale is used, for example with numbers from 1 to 10, where 1 is a “bad” prediction index (a status of the patient will probably deteriorate and even die if nothing is done, as shown in reference profiles located in the reference database), and 10 is a “good” prediction index (a status of the patient will improve and/or not change, as shown in reference profiles located in the reference database).

In some embodiments, a wording scale is used. For example, when a status of a patient is good, the prediction index will be “sporadic monitoring is needed”, while when a status of a patient is bed, the prediction index will be “frequent monitoring is need and actions are to be taken immediately”. It should be understood that these are just examples and that any wording used are included in the scope of the invention.

### EXAMPLE

An exemplary prediction index comprises a scale of 1 to 10 with weekly resolution that is meant to track a heart failure patient's cardiac functional elements. In some embodiments, the results of the assessments are provided to physicians to assess changes in functional elements to evaluate, for example, the effect of therapy (CCM and others) and guide treatment to prevent decompensation and hospitalizations that would be predicted by the index. In some embodiments, the results of the assessments are provided to patients to use as a warning sign of worsening so they can make lifestyle corrections to keep them out of the hospital, as well as a motivator for sustaining therapy (e.g., recharging the implantable device, not missing medications) and maintaining a healthy lifestyle (e.g., diet, exercise, reduced salt intake, etc.).

In some embodiments, variations in the personalized prediction index throughout time is used and provided as risk-classification parameters suitable for the prediction of HF-related hospitalizations and mortality.

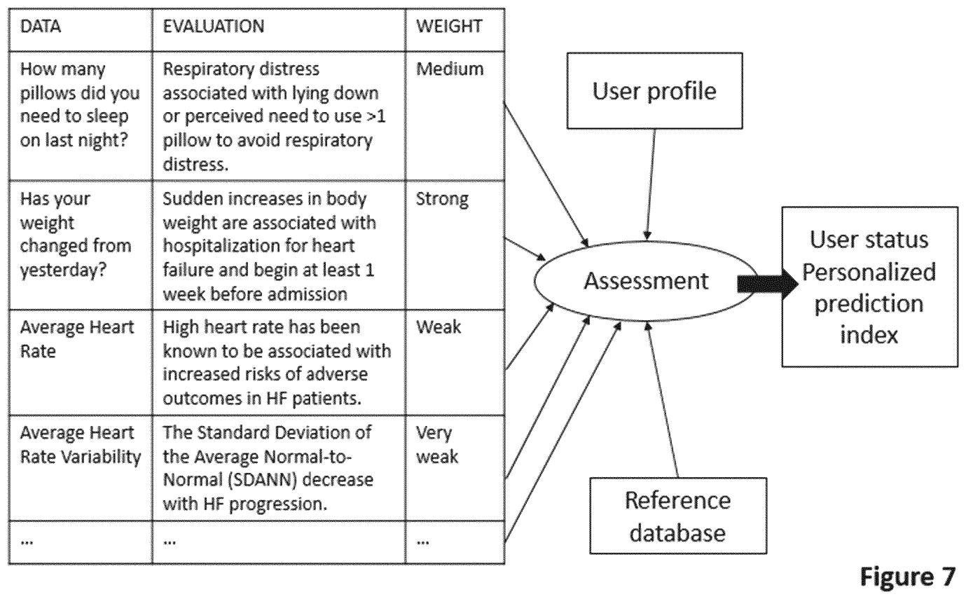

Referring now to FIG. 7, showing a schematic representation of an exemplary process of monitoring a patient, according to some embodiments of the invention. FIG. 7 shows a table having exemplary data that is inserted into the system, for example the average heart rate. The table also shows the evaluation according to the response provided, in the example of average heart rate, increase in heart rate is associated with increased risks in HF patients. The table also show the weight that has been provided to that specific data, in this example it is “weak”, which means that a change in that specific parameters does not influence much in the overall status of the patient. In some embodiments, the plurality of data, evaluation and weights are delivered for assessment, where in conjunction with the user profile and the reference database, the user current status and personalized prediction index are recovered.

As used herein with reference to quantity or value, the term “about” means “within +20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for monitoring a status of a user, comprising:
a. an implantable device, configured to communicate with an external electronic device;
b. an external electronic device, comprising:
i. a generated reference database;
ii. an assessment module comprising instructions to generate a personalized prediction index; and
iii. a user database comprising user data;
wherein said implantable device is an Implantable Cardioverter-Defibrillator (ICD); and wherein said ICD is configured to perform one or more of monitoring the cardiac activity, providing Cardiac resynchronization therapy (CRT) and providing Cardiac contractility modulation (CCM).

2-3. (canceled)

4. The system according to claim 1, wherein said generated reference database is generated according to a method comprising:
a. collecting historical data from a plurality of users; said historical data comprises a chronological timeline;
b. indexing said collected data;
c. analyzing said indexed data;
d. grouping said analyzed indexed data according to similarities at a beginning of said chronological timeline;
e. analyzing each of said analyzed groups to recover similarities and differences in the outcomes of said plurality of users;
f. identifying diverging points in said chronological timeline;
g. providing indexing values at said diverging points of said analyzed chronological timeline;
h. storing said indexing values in a database for use as reference prediction index.

5. The system according to claim 1, wherein said personalized prediction index is generated according to a method comprising:
a. collecting a plurality of data related to a plurality of different users;
b. generating a plurality of reference prediction indexes based on said collected plurality of data;
c. collecting data related to a determined user;
d. comparing said collected data related to said determined user with said reference prediction indexes;
e. generating a personalized prediction index according to said comparing.

6. The system according to claim 1, wherein said external electronic device is a server.

7. The system according to claim 1, wherein said external electronic device is an electronic device comprising instructions for delivering data to a server.

8. The system according to claim 1, wherein said external electronic device comprises a dedicated software comprising instructions to allow said user to insert user data;
wherein said dedicated software comprises instructions for providing said user with a dedicated questioner.

9. (canceled)

10. The system according to claim 1, further comprising one or more external sources for providing data related to said user.

11. The system according to claim 1, wherein said personalized prediction index is used to predict an expected change in said status of said user.

12. A method of generating a personalized prediction index, comprising:
a. collecting a plurality of data related to a plurality of different users;
b. generating a plurality of reference prediction indexes based on said collected plurality of data;
c. collecting data related to a determined user;
d. comparing said collected data related to said determined user with said reference prediction indexes;
e. generating a personalized prediction index according to said comparing.

13. The method according to claim 12, wherein said data related to a determined user comprises one or more of user medical history, clinical parameters, data from an implantable device and data from external sensors; and
wherein said external sensors are one or more of ECG sensor, Blood pressure sensor, Bioimpedance sensor, Respiratory sensor, Movement sensor and Sleep sensor.

14. (canceled)

15. The method according to claim 12, wherein said collecting data related to a determined user comprises collecting data from one or more of user himself, one or more sensors connected to said user and one or more external sources; and
wherein said collecting data from the user himself comprises providing said user with a dedicated questioner.

16. (canceled)

17. The method according to claim 12, wherein said personalized prediction index is used to predict an expected change in a status of said user.

18. A method of monitoring a status of a user, comprising:
a. generating a personalized prediction index;
b. collecting one or more status data of said user;
c. updating said personalized prediction index according to said collected status data.

19. The method according to claim 18, wherein said generating a personalized prediction index comprises:
a. collecting a plurality of data related to a plurality of different users;
b. generating a plurality of reference prediction indexes based on said collected plurality of data;
c. collecting data related to a determined user;
d. comparing said collected data related to said determined user with said reference prediction indexes;
e. generating a personalized prediction index according to said comparing.

20. The method according to claim 19, wherein said data related to a determined user comprises one or more of user medical history, clinical parameters, data from an implantable device and data from external sensors;
wherein said external sensors are one or more of ECG sensor, Blood pressure sensor, Bioimpedance sensor, Respiratory sensor, Movement sensor and Sleep sensor; and
wherein said collecting data related to a determined user comprises collecting data from one or more of user himself, one or more sensors connected to said user and one or more external sources; wherein said collecting data from the user himself comprises providing said user with a dedicated questioner.

21-23. (canceled)

24. The method according to claim 18, wherein said personalized prediction index is used to predict an expected change in said status of said user; and further comprising analyzing said collected data after said collecting and before said updating;
wherein said analyzing comprises comparing said collected data with one or more reference data of one or more reference prediction indexes.

25-26. (canceled)

27. The method according to claim 18, further comprising updating at least one external entities with said updated personalized prediction index.

28. The method according to claim 18, further comprising sending an alert when a change in said personalized prediction index reaches a predetermined threshold.

29. The method according to claim 18, wherein said collecting one or more status data of said user comprises collecting data from one or more of user himself, one or more sensors connected to said user, one or more external sources; and
wherein said collecting data from the user himself comprises providing said user with a dedicated questioner.

30. (canceled)

31. The method according to claim 18, wherein said updating said personalized prediction index is used for predicting an expected change in said status of said user.

32-33. (canceled)
